(19) United States
(12) Patent Application Publication
(10) Pub. No.: US 2010/0305480 A1
(43) Pub. Date: Dec. 2, 2010

(54) Human Motion Classification At Cycle Basis Of Repetitive Joint Movement

(76) Inventors: Guoyi Fu, Toronto (CA); Mark Christopher Jeffrey, Toronto (CA)

Correspondence Address: EPSON RESEARCH AND DEVELOPMENT INC;INTELLECTUAL PROPERTY DEPT, 2580 ORCHARD PARKWAY, SUITE 225, SAN JOSE, CA, 95131, US

(21) Appl. No.: 12/475,809

(22) Filed: Jun. 1, 2009

(51) Int. Cl. A61B 5/11

(52) U.S. Cl. 600/595

(57) ABSTRACT

Methods and systems for classifying human motion as corresponding to an activity are disclosed. One example method includes sensing motion characteristics associated with the activity to generate a first set of data, identifying a cycle interval in the first set of data; and identifying the activity based on the interval.

### THE FIELD OF THE INVENTION

Embodiments of the invention relate to classifying human motion at a cycle basis of repetitive joint movement. More specifically, disclosed embodiments relate to methods, devices, and computer-readable media for recognizing human motion and classifying the motion as corresponding to a particular activity.

### BACKGROUND

Physical inactivity is known to contribute to many chronic diseases, such as cardiovascular diseases, type-2 diabetes, and many other health risks. To combat such risks, moderate intensity physical workouts are recommended to achieve a basic level of physical activity to manage weight, lower blood pressure, and improve sugar tolerance, among other things. The level of daily physical activity can be measured objectively by measuring energy expenditure. In addition to the quantitative daily energy expenditure, qualitative activity types play an important role in overall well being and health. Automatic classification of daily activities or motions can be used for promotion of healthier lifestyle or for daily physical activity monitoring. Furthermore, activity classification can improve accuracy of energy expenditure estimations.

### SUMMARY OF EXAMPLE EMBODIMENTS

In general, example embodiments relate to methods, devices, and computer-readable media for classifying human motion as corresponding to a specific type or category of physical activity.

In a first example embodiment, a method for classifying human motion as corresponding to a physical activity includes sensing motion characteristics associated with the activity to generate a first set of data, identifying a cycle interval in the first set of data; and then identifying the type of activity based at least in part on the interval.

In another example embodiment, a method for assessing fitness of a human subject is disclosed. In a disclosed example, this includes sensing characteristics associated with physical activities using one or more sensors attached to the body and then identifying a cyclical pattern in at least one of the sensed characteristics. The physical activity or activities are then identified based at least in part on the sensed characteristics and the cyclical pattern. The information can then be used to assess the fitness of the subject and/or otherwise used to monitor health, track performance of a particular exercise routine, build medical histories, detect health risks, and/or augment a virtual reality system, among other things.

In yet another example embodiment, one or more computer-readable media have computer-readable instructions thereon which, when executed, implement all or portions of the method for activity classification discussed above in connection with the first example embodiment.

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

Additional features will be set forth in the description which follows, and in part will be obvious from the description, or may be learned by the practice of the teachings herein. Features of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

To further develop the above and other aspects of example embodiments of the invention, a more particular description of these examples will be rendered by reference to specific embodiments thereof which are disclosed in the appended drawings. It is appreciated that these drawings depict only example embodiments of the invention and are therefore not to be considered limiting of its scope. It is also appreciated that the drawings are diagrammatic and schematic representations of example embodiments of the invention, and are not limiting of the present invention. Example embodiments of the invention will be disclosed and explained with additional specificity and detail through the use of the accompanying drawings in which:

FIG. 1 discloses an example method for classifying human motion as corresponding to an activity;

FIG. 2 is a graphical representation of an example environment in which the method of FIG. 1 may be performed;

FIG. 3 discloses a schematic representation of an example portable computing device for use in performing the method of FIG. 1;

FIG. 4 discloses an example decision tree representing an order of activity identification performed in the method of FIG. 1;

FIG. 5 discloses example graphs of angular velocity data gathered by a gyroscopic sensor attached to an ankle during different activities;

FIGS. 6A-6E disclose graphs of data used to identify events and intervals for use in identifying the activity, each figure corresponding to a different activity;

FIG. 7 discloses an example swing cycle interval in an example graph of angular velocity data; and

FIGS. 8A-8D disclose graphs of motion data in various motion feature spaces used to distinguish different activities from each other.

### DETAILED DESCRIPTION OF EXAMPLE EMBODIMENTS

In the following detailed description, reference is made to the accompanying drawings that show, by way of illustration, example embodiments of the invention. In the drawings, like numerals describe substantially similar components throughout the several views. These embodiments are described in sufficient detail to enable those skilled in the art to practice the invention. Other embodiments may be utilized and structural, logical and electrical changes may be made without departing from the scope of the present invention. Moreover, it is to be understood that the various embodiments of the invention, although different, are not necessarily mutually exclusive. For example, a particular feature, structure, or characteristic described in one embodiment may be included within other embodiments. The following detailed description is, therefore, not to be taken in a limiting sense, and the scope of the present invention is defined only by the appended claims, along with the full scope of equivalents to which such claims are entitled.

In general, example embodiments relate to methods, devices, and computer-readable media that can be used to classify human motions as corresponding to a particular physical activity, such as different types of exercise. Example embodiments can be used in conjunction with a personal or body area network to monitor health, track performance of an exercise routine, build medical histories, detect health risks, and/or augment a virtual reality system, among other things.

In performing many physical activities and exercises, a cyclical or repetitive motion occurs. A gyroscopic signal from a sensor attached to the human body may be used to capture characteristics of, for example, repetitive joint rotation. According to one disclosed example embodiment, a gyroscopic sensor may be attached to an ankle to capture data characterizing movement of a shank portion of the body during, for example, walking, running, cycling, rowing, and elliptical walking, among other activities. A cyclical pattern may be identified in angular rotation velocity data generated by, or derived from data generated by, the gyroscopic sensor. A specific type of activity can be identified based, at least in part, on distinguishable features extracted from the cyclical pattern or from other motion data gathered by other sensors, using the cyclical pattern as a reference.

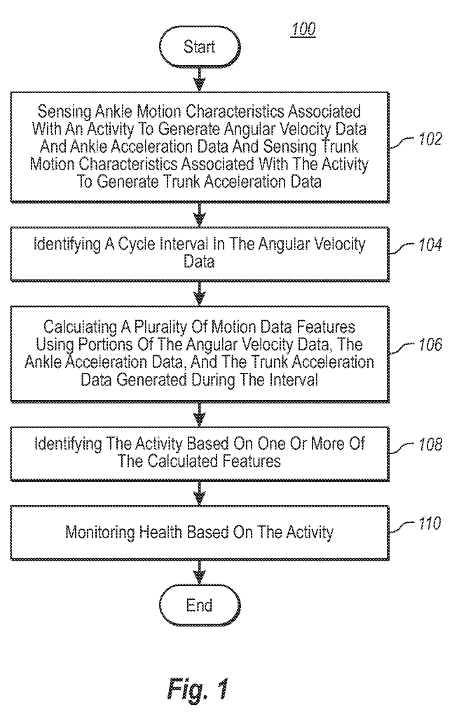

With reference now to FIG. 1, one example of a series of steps used in a method 100 for classifying human motion as corresponding to a particular activity is disclosed. The example method 100 identifies the type of activity based, at least in part, on features extracted or derived from data gathered by one or more sensors positioned at predetermined points on a human body that is engaged in the activity.

The example method 100 and variations thereof that are disclosed herein can be implemented by way of one or more computer-readable media configured to carry or otherwise have computer-executable instructions or data structures stored thereon. Such computer-readable media can be any available media that can be accessed by a processor of a general purpose or special purpose computer. By way of example and not limitation, such computer-readable media can comprise RAM, ROM, EEPROM, CD-ROM or other optical disk storage, magnetic disk storage or other magnetic storage devices, or any other medium which can be used to carry or store program code in the form of computer-executable instructions or data structures and which can be accessed by a processor of a general purpose or special purpose computer. Combinations of the above should also be included within the scope of computer-readable media.

Computer-executable instructions comprise, for example, instructions and data which cause a processor (or similar programmable logic) of a general purpose computer or a special purpose computer to perform a certain function or group of functions. Although the subject matter described herein is presented in language specific to methodological acts, it is to be understood that the subject matter defined in the appended claims is not necessarily limited to the specific acts described herein. Rather, the specific acts described herein are disclosed as example forms of implementing the claims.

Examples of special purpose computers can include portable computing devices such as personal digital assistants (PDAs), handheld computing devices, cellular telephones, laptop computers, audio/video media players, or combinations thereof. A portable computing device may be part a of a personal area network that also includes sensors gathering motion data and feeding the data to the portable computing device for processing. The computing device may include an activity classification capability to, for example, monitor and gauge health risks and improvements, track performance of an exercise routine, build medical histories, and/or augment a virtual reality system, among other things. For example, a computing device with this activity classification capability may include one or more computer-readable media that implement the example method 100 and may send results to physical trainers, emergency personnel, caregivers, physicians, a medical history database, and/or a virtual reality display. Alternatively, a computer connected to and in communication with the computing device via a network connection may include one or more computer-readable media that implement the example method 100. The connected computer may send results to a portable computing device and/or to a trainer, emergency personnel, caregivers, physicians, a medical history database, and/or a virtual reality display.

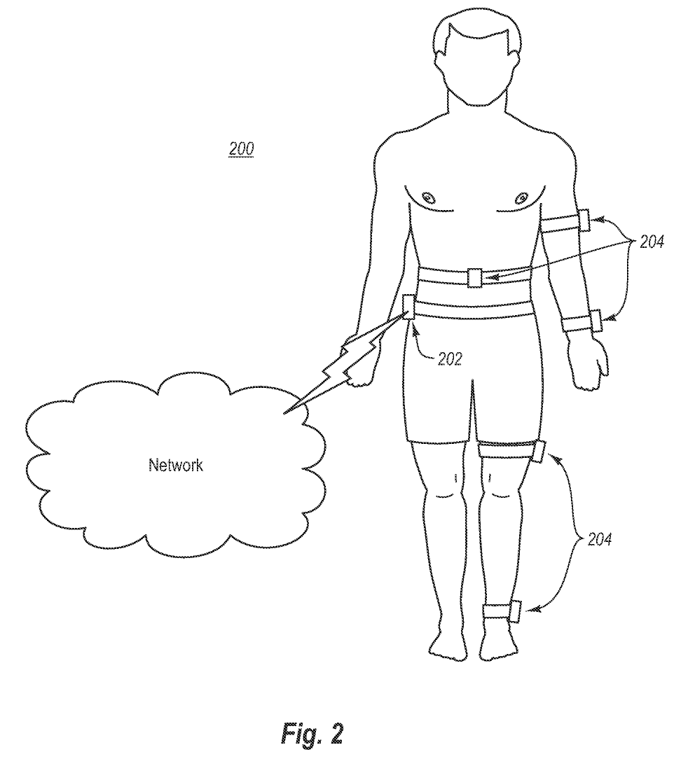

FIG. 2 discloses a graphical representation of one example of a personal area network 200 that might be used in connection with the example method of FIG. 1. As is shown in the example, the personal network 200 includes a portable computing device 202. The personal area network 200 may also include one or more sensors, denoted at 204, which can be operably connected to the portable computing device 202 for communication. Operative connections between the sensors 204 and the portable computing device 202 can be implemented by way of wired connections, or the personal area network 200 may utilize wireless protocols such as Bluetooth or ZigBee. The sensors 204 may include motion sensors, such as accelerometers, tilt sensors, and gyroscopic sensors, as well as other types of sensors. For example, sensors that monitor the environment such temperature sensors and humidity sensors might be used. Also, sensors that monitor physiological parameters of the subject, such as heart rate sensors, blood pressure sensors, and the like might be used. The sensors 204 may be attached at various places on the subject, including wrist, bicep, waist/trunk, thigh, and ankle areas, using any suitable fastener, such as a hook and loop fastener (e.g., Velcro®-brand fasteners). Alternatively, one or more of the sensors 204 may be integrated within clothing worn by the subject. In one example, the portable computing device 202 may include an Xbus Master, manufactured by Xsens Technologies (www-xsens-com), with a Bluetooth wireless link to each of the sensors 204. In addition, the sensors 204 may be, for example, an MTx sensing device included in an Xbus kit and manufactured by Xsens Technologies, which includes the Xbus Master. Of course, other types of “orientation” tracking devices could also be used.

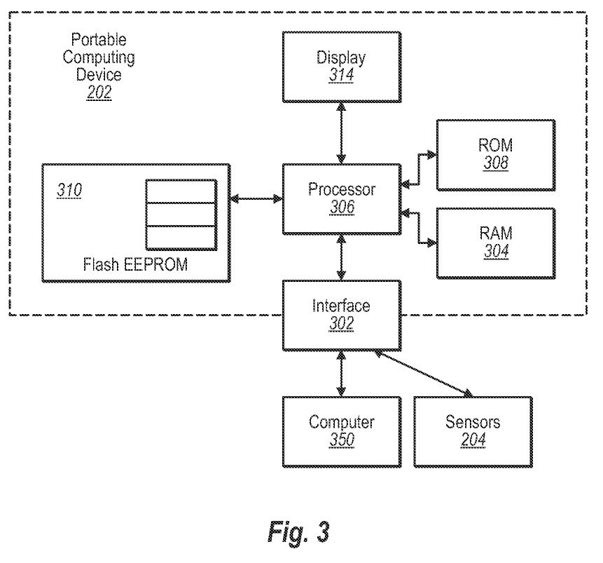

FIG. 3 discloses a more detailed schematic representation of one example of a portable computing device, denoted at 202, that might be used in connection with disclosed embodiments. In this illustrated example, the portable computing device 202 exchanges data with another computer 350 and with the sensors 204 by way of an interface 302. Application programs and data may be stored for access on the computer 350. The network interface 302 may be a network interface and/or may be implemented as either a wired or wireless interface (or a combination).

When data is received from the computer 350 or the sensors 204, the interface 302 receives the data and stores it in a receive buffer forming part of a RAM 304. While different memory access and storage arrangements might be used, in the illustrated example the RAM 304 is divided into a number of sections, for example through addressing, and logically allocated as different buffers, such as a receive buffer or a send buffer. Data, such as motion data or application programs, can also be obtained by the portable computing device 202 from the flash EEPROM 210, or the ROM 208.

A programmable processor 306 uses computer-executable instructions stored on a ROM 308 or on a flash EEPROM 310, for example, to perform a certain function or group of functions, such as the method 100 for example. Where the data in the receive buffer of the RAM 304 is motion data received from one or more of the sensors 204, for example, the processor 306 can implement the methodological acts of the method 100 on the motion data to detect a cyclically occurring interval and to identify an activity based on the identified interval as well as based on features of the motion data. The features may be derived using the interval and/or motion events occurring in the interval as a timing reference. Further processing may be performed on the motion data, if necessary, and an indication of the identified activity (e.g., a graphic and/or text) may be displayed by the portable computing device 202 on a display 314, such as an LCD panel, for example, or transferred to the computer 350.

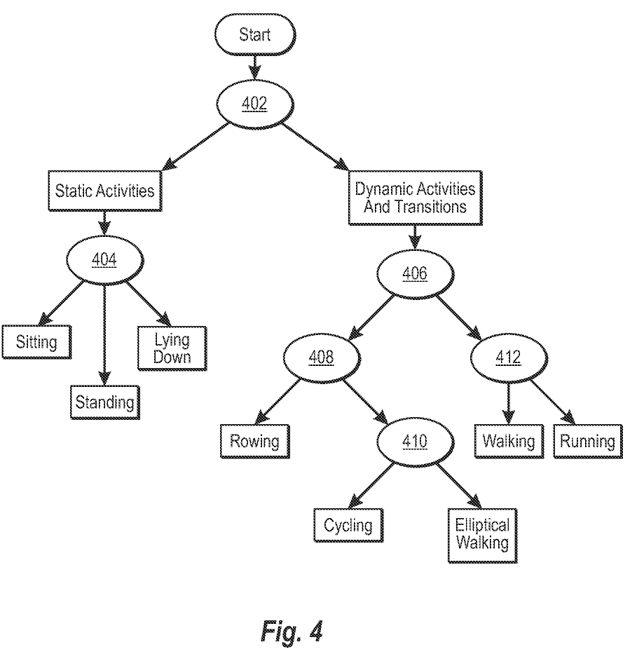

FIG. 4 discloses an example decision tree representing an order of activity identification performed in the method 100. FIG. 4 shows a few examples of the different types of physical activities or exercises that might be recognized by performance of the method 100. Activities that may be identified include static activities, dynamic activities, and/or transition activities (i.e., moving between static and dynamic).

Static activities may be distinguished from dynamic and transition activities at node 402 by, for example, determining whether the sensors are in a static state or if one or more of the sensors indicate significant body movement. Such determinations may be made based on orientation data, acceleration data, angular velocity data, temperature data, and/or heart rate data received from the sensors 204. Static activities may be further identified at node 404 as, for example, sitting, standing, and lying down.

Dynamic activities, on the other hand, may include walking, running, cycling, etc. For example, in embodiments discussed herein, the dynamic activities recognized by the method 100 include walking, running, cycling, elliptical machine walking (i.e., elliptical walking), and rowing, any one of which may be performed with or without the aid of an exercise machine, such as a treadmill or stationary bicycle. Distinctions between each of the foregoing activities are made at nodes 406-412. More complex dynamic activities, e.g., playing sports such as tennis or soccer, may be recognized as a combination of primitive dynamic activities, such as running, walking, swinging arms, kicking, etc.

The example method 100 for classifying human motion as corresponding to an activity will now be discussed in connection with FIG. 1. At 102, ankle motion characteristics and trunk motion characteristics associated with an activity are sensed. The ankle motion characteristics can be sensed using an accelerometer and a gyroscopic sensor to generate ankle acceleration data and angular velocity data, respectively. The trunk motion characteristics can be sensed using an accelerometer to generate trunk acceleration data. The ankle acceleration data, angular velocity data, and trunk acceleration data can be generated at the same time and can be synchronized. Before using the data to identify the activity, however, the angular velocity data can first be analyzed to identify a cycle interval (e.g., a swing cycle interval) at 106.

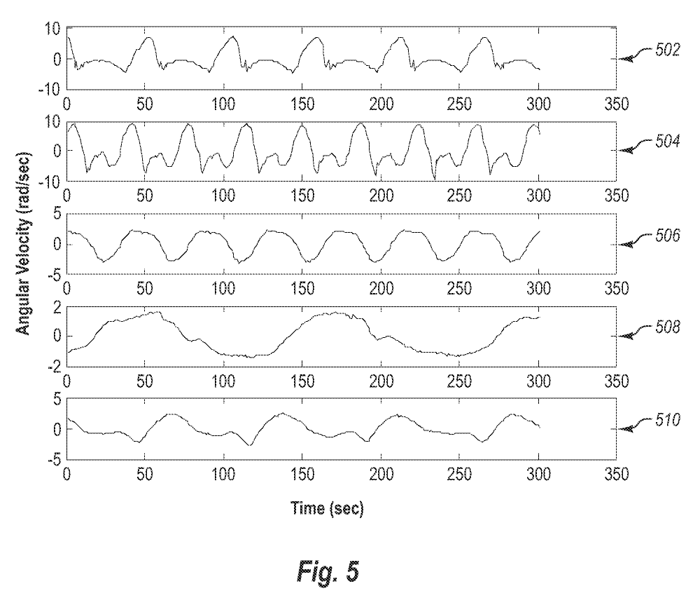

FIG. 5 discloses various example graphs of angular velocity data gathered by a gyroscopic sensor attached to an ankle during different activities. A gyroscopic sensor can sense angular velocity in three dimensions or axes. The angular velocity data in each graph of FIG. 5 can correspond to the axis sensing the largest or dominant angular velocity, which in the examples shown, corresponds to a pivot axis of the ankle to which the sensor is attached.

For example, the graph 502 depicts angular velocity data generated during a three mile per hour walking activity. The graph 504 depicts angular velocity data generated during a seven mile per hour running activity. The graph 506 depicts angular velocity data generated during a seventy revolutions per minute cycling activity. The graph 508 depicts angular velocity data generated during a forty-five revolutions per minute elliptical walking activity. As shown in each graph, each activity is characterized by a periodic positive (i.e., forward) swing event. A rotation angle derived from each set of angular velocity data can be used to identify the forward swing event, a swing cycle period or interval, and a forward swing phase or interval (i.e., duration of a forward swing event), among other things.

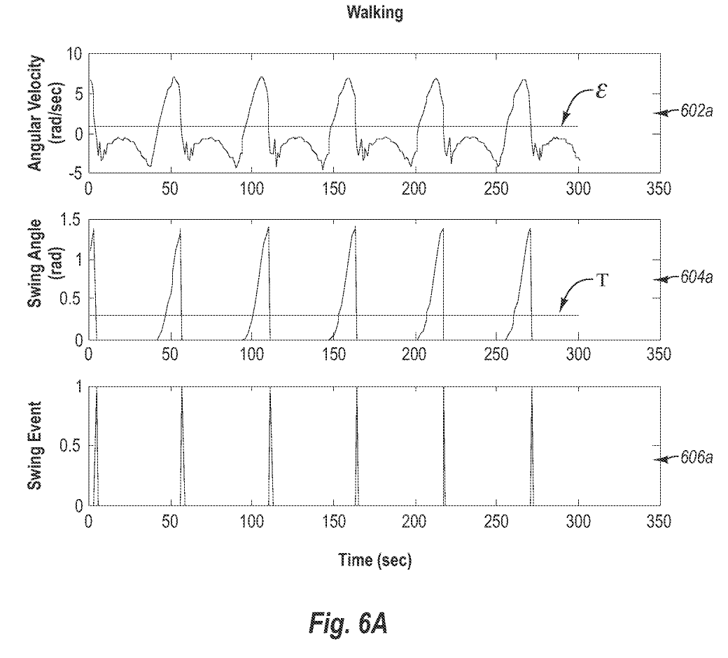

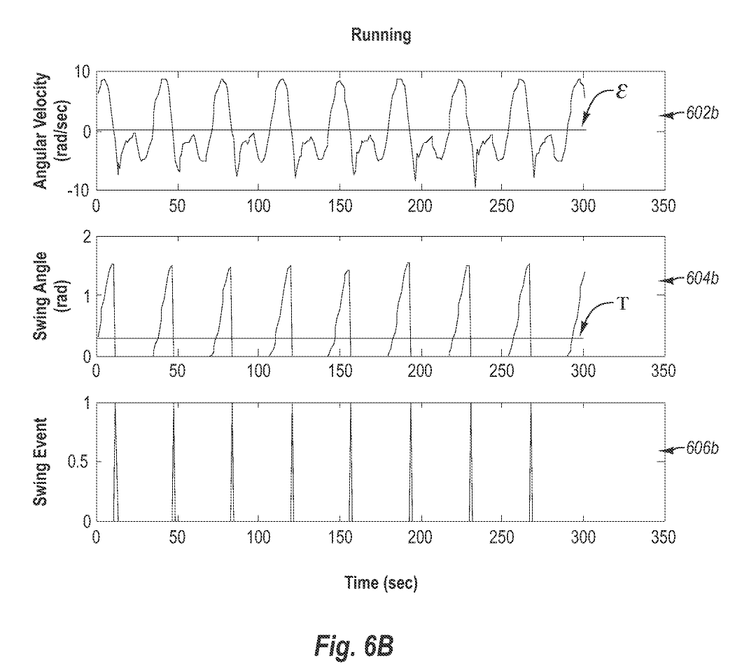

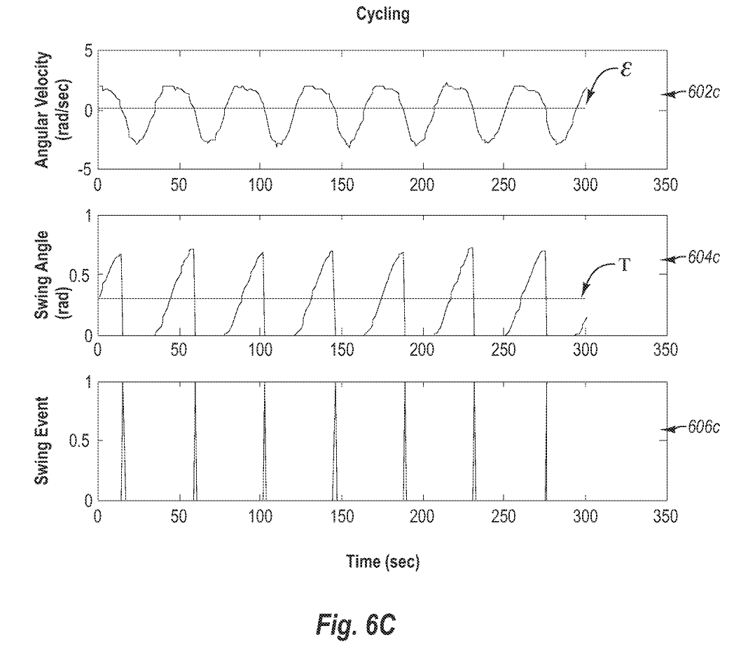

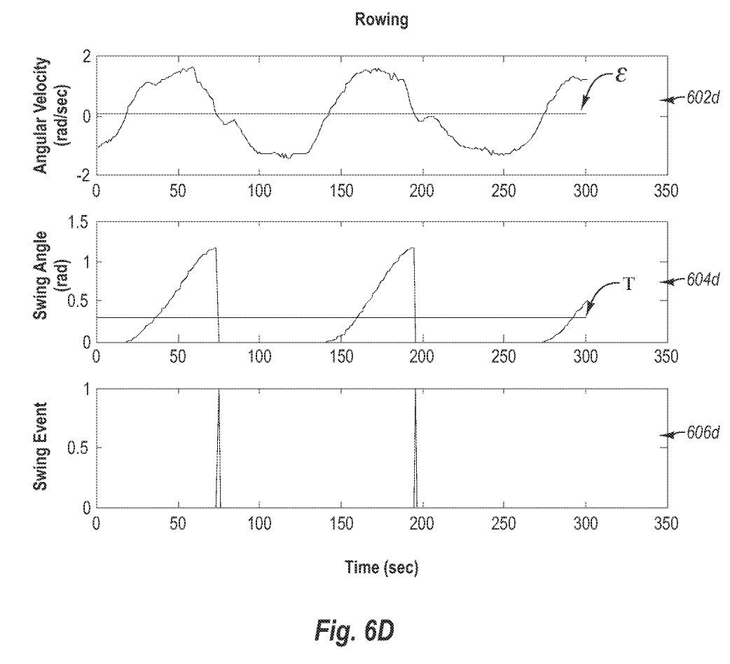

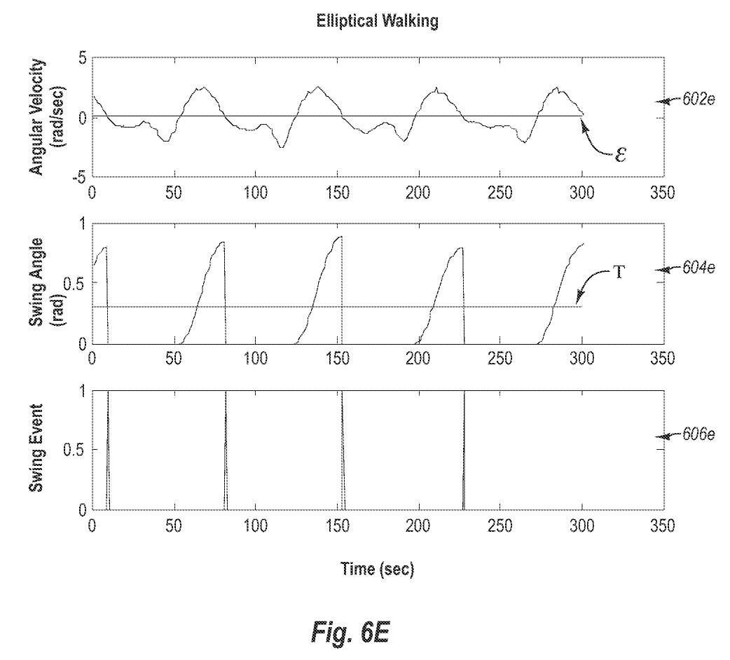

FIGS. 6A-6E disclose graphs having data points that can be used to identify swing events, a swing cycle interval, and a forward swing phase or interval within the swing cycle interval. Swing events, corresponding to a step, stride, or cycle, may define swing cycle intervals in the angular velocity data. Each of FIGS. 6A-6E is labeled as corresponding to a different dynamic activity. For example, graph 602a in FIG. 6A shows angular velocity data generated while a subject is walking, FIG. 6B corresponds to data generated while running, FIG. 6C to cycling, FIG. 6D to rowing, and FIG. 6E to elliptical walking. The identification of swing events and intervals is similar for each activity and will therefore be described without reference to a particular activity.

To identify a swing event, portions of the angular velocity data exceeding a significance threshold level may first be integrated to generate swing angle data, e.g., as follows:

$$Swing Angle ( i ) = \{ \begin{matrix}Swing Angle ( i - 1 ) + Gz ( i ) / Sampling Frequency & Gz ( i ) > ɛ \\ Swing Angle ( i ) = 0 & Gz ( i ) <= ɛ\end{matrix}$$

where Swing Angle(i) is a swing angle value at time i, Gz(i) is an angular velocity value from a gyroscopic sensor generated at time i and corresponding to angular velocity about an axis with a dominant proportion of rotation (e.g., a joint pivot axis), Sampling Frequency is a frequency at which the angular velocity data is sampled, and ε is a threshold value. A graph 604a shows the swing angle data derived from the angular velocity data in the graph 602a according to the swing angle formula above. (Corresponding graphs 604b, 604c, 604d, and 604e are shown in FIGS. 6B through 6E for comparison.) The threshold value ε can be set to eliminate consideration of insignificant levels of angular velocity caused by phenomena such as gyroscopic sensor drift or noises.

In the swing angle formula above, the threshold value ε can be constrained to be a positive value that is small relative to a peak positive swing angle velocity. Integration of only a positive or forward swing angle can be performed because a forward swing velocity is, for many activities (e.g., running and walking), of a greater magnitude and therefore more easily detectable than a negative or backward swing velocity. However a backward swing angle could also be a basis for cycle identification, either instead of or in combination with the forward swing angle, particularly for activities where the backward swing angle velocity is more pronounced. Thus, although the embodiments described herein use a forward swing angle, a backward swing angle could also be used.

With the derivation of the swing angle data, swing events may be identified by falling edges of the swing angle data in the graph 604a. To avoid false detection due to noise or vibrations, a threshold T can be used as follows:

$$Swing Event ( i ) = \{ \begin{matrix}1 & Swing Angle ( i - 1 ) > T AND Swing Angle ( i ) \leq T \\ 0 & otherwise\end{matrix}$$

Graphs 606a, 606b, 606c, 606d, and 606e each show identification of swing events using the swing event formula above. The swing event formula can be modified to detect other features of the swing angle data as swing events, e.g., a rising edge, a peak, etc., so long as an interval of time between swing events is identified.

Referring again to FIG. 1, at 108 an act of calculating a plurality of motion data features using portions of the angular velocity data, the ankle acceleration data, and the trunk acceleration data is performed. Because the angular velocity data in a swing cycle interval corresponding to each activity has distinct features, as shown in FIGS. 6A-6E, the portions of angular velocity data used to calculate motion data features might be limited to portions generated during a swing cycle interval. Moreover, portions of other motion data, such as ankle acceleration data and the trunk acceleration data, used to calculate motion data features can be limited to portions generated during a swing cycle interval.

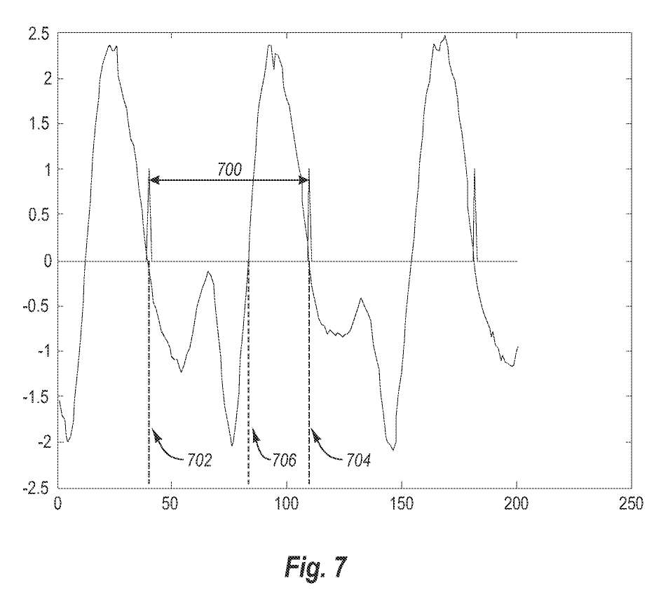

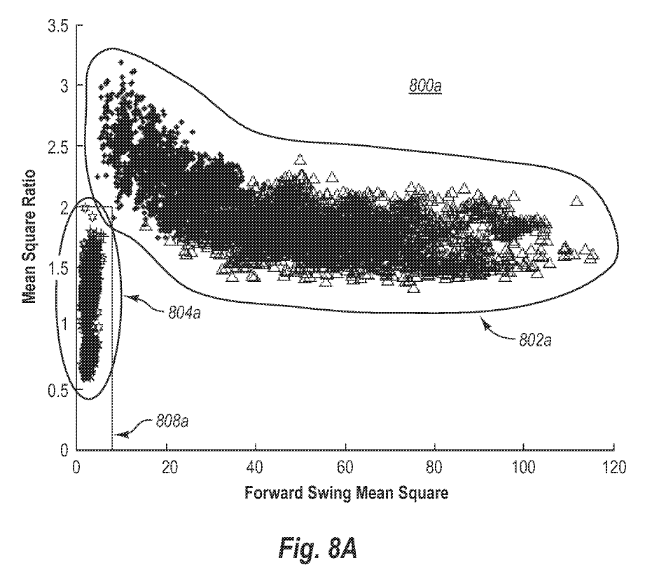

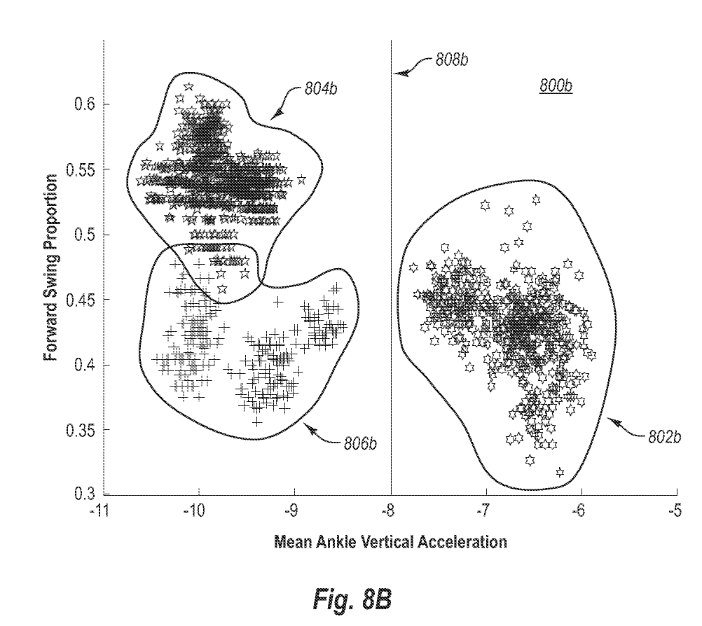

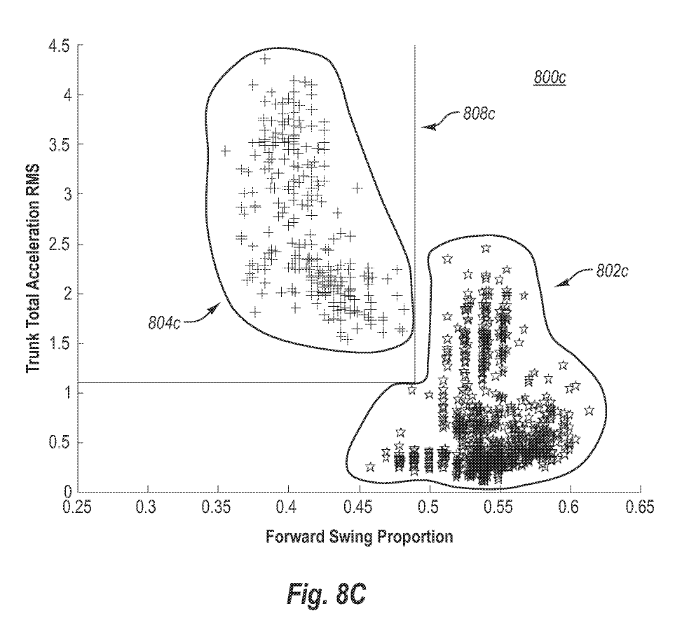

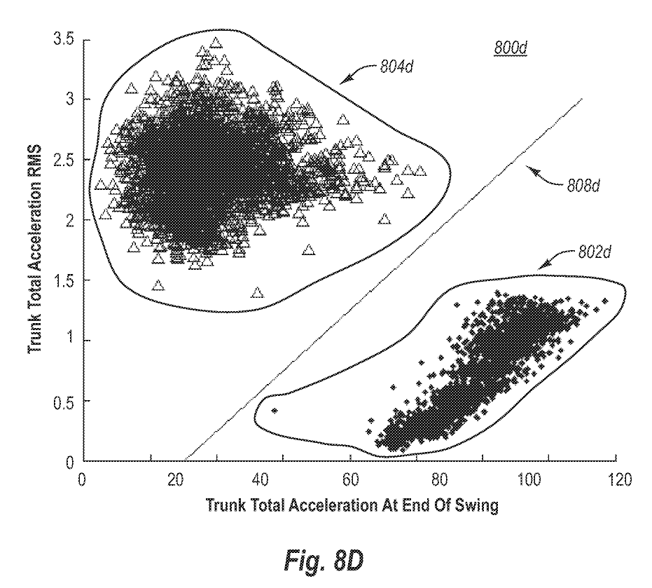

FIG. 7 discloses an example swing cycle interval 700 in an example graph of angular velocity data. Various timing aspects of the interval 700 can be used in deriving or extracting motion data features from the angular velocity data as well as from other motion data. For example, an interval start time 702 (ts) and an interval end time 704 (te) define a duration of the swing cycle interval 700. Moreover, a forward swing starting time 706 (tp) and the end time 704 define a forward swing interval (i.e., an interval in which angular velocity is positive) within the swing cycle interval 700. These intervals of time can be used to delimit portions of motion data used to extract motion data features, as explained in more detail below with reference to FIGS. 8A-8D. The end time 704 is shown as corresponding to both the end time of the swing cycle interval and the forward swing interval. However, the end time 704 and start time 702 of the swing cycle interval 700 might be shifted in either direction, so long as the interval is held constant. For example, if peak swing velocity events are identified and used to define the swing cycle interval instead of forward swing events, the start time 702 and end time 704 would correspond to a peak in the angular velocity data instead of a zero crossing.

FIGS. 8A-8D disclose graphs of motion data in various motion feature spaces used to distinguish different activities from one another. Each graph of FIGS. 8A-8D shows empirically derived data points corresponding to various dynamic activities in a feature space defined by a different motion feature on each axis. Each graph also shows one or more threshold lines used to distinguish the various activities. The threshold lines represent decision criteria applied by the classification decision nodes 406-412 in FIG. 4.

FIG. 8A shows a feature space 800a that is defined by a forward swing mean square feature, on the x-axis, and a mean square ratio feature, on the y-axis. Data points 802a correspond to a walking activity (diamonds) or running activity (triangles), whereas data points 804a correspond to a rowing (six-pointed stars), cycling (five-pointed stars), or elliptical walking (crosses) activity. The forward swing mean square feature, which is the x-axis of the feature space 800a, may be defined as follows:

$$Forward Swing Mean Square = \frac{{\sum }_{i = tp}^{te} {Gz ( i )}^{2}}{te - tp}$$

where Gz(i) is an angular velocity value generated at time i by a gyroscopic sensor attached to an ankle and corresponding to angular velocity about a dominant pivot axis. The root of the forward mean square feature represents a magnitude of angular velocity during a forward swing period, but to avoid the computational cost of a square root calculation the forward swing mean square can instead be used.

The mean square ratio feature, which is the y-axis of the feature space 800a, may be defined as follows:

$$Mean Square Ratio = \frac{Forward Swing Mean Square}{Cycle Mean Square}$$

where the cycle mean square is the defined as follows:

$$Cycle Mean Square = \frac{{\sum }_{i = ts}^{te} {Gz ( i )}^{2}}{te - ts}$$

The root of the mean square ratio represents a ratio of a magnitude of angular velocity during a forward swing interval (from tp to te) to a magnitude of angular velocity during an entire swing cycle interval (from ts to te). Here again, to avoid the computational cost of a square root calculation, a mean square ratio may be used instead of a root mean square (RMS) ratio.

The features defining the feature space 800a are useful in distinguishing running and walking, on the one hand, from rowing, cycling, and elliptical walking, on the other hand. The activities may be distinguished in the feature space 800a by the following decision criteria:

$$Activity = \{ \begin{matrix}walking or running & \begin{matrix}Mean Square Ratio > 2 AND \\ Forward Swing Mean Square > 9\end{matrix} \\ \begin{matrix}cycling , rowing , or \\ elliptical walking\end{matrix} & otherwise\end{matrix}$$

The foregoing decision criteria is shown as a threshold 808a in the feature space 800a. The threshold 808a effectively distinguishes a forward swing motion performed in the air from a forward swing motion performed along the path of a pedal. For example, an in-air ankle swing frequently has a larger angular velocity during the forward swing phase, particularly when the ankle is engaged in running, than does a pedaling (i.e., along the path of a pedal) ankle swing. Thus, the forward swing mean square is useful in distinguishing between these two types of swinging. However, a very slow walking activity could also have a small forward swing mean square due to a small angular velocity during the forward swing. Therefore the mean square ratio feature might also be used to distinguish walking from pedaling activities. The mean square ratio feature will be higher for slow walking than for other activities because slow walking often includes a longer period of standing (i.e., zero velocity) outside of the forward swing phase. In short, because an in-air ankle swing will frequently have either a faster forward swing or a higher ratio of forward swing magnitude to total swing magnitude than a pedaling ankle swing, the feature space 800a is an effective space in which to distinguish walking and running, which involve in-air ankle swings, from rowing, cycling, and elliptical walking, which involve pedaling ankle swings.

FIG. 8B shows a feature space 800b that is defined by a mean ankle vertical acceleration feature on the x-axis, and a forward swing proportion feature on the y-axis. In the feature space 800b, data points 802b (six-pointed stars) correspond to a rowing activity, data points 804b (five-pointed stars) correspond to a cycling activity, and data points 806b (crosses) correspond to an elliptical walking activity. The mean ankle vertical acceleration, which is the x-axis of the feature space 800b, feature may be defined as follows:

$$Mean Ankle Vertical Acceleration = \frac{{\sum }_{i = te}^{tp} Av ( i )}{te - ts}$$

where Av(i) is an acceleration value generated at time i by an accelerometer attached to an ankle and corresponding to acceleration in a vertical direction. The forward swing proportion feature, which is the y-axis of the feature space 800b, may be defined as follows:

$$Forward Swing Proportion = \frac{te - tp}{te - ts}$$

The features defining the feature space 800b are useful in distinguishing rowing, cycling, and elliptical walking from each other. In particular, the mean ankle vertical acceleration feature can be used to distinguish a rowing activity, on the one hand, from cycling and elliptical walking activities, on the other. When rowing, the ankle swings between an upright to horizontal position, whereas during cycling and elliptical walking the ankle generally remains in an upright position. Thus, an accelerometer attached to the ankle would experience less gravity in its vertical direction on average when rowing than when cycling or elliptical walking. Thus the mean ankle vertical acceleration feature is a useful feature for distinguishing a rowing activity from a cycling or elliptical walking activity.

As shown, in the feature space 800b, the forward swing proportion feature is another useful feature that can be used to distinguish between different activities. However, according to one embodiment, activities may be distinguished in the feature space 800b using only the mean ankle vertical acceleration feature according to the following decision criteria, which is represented by a threshold 808b in the feature space 800b:

$$Activity = \{ \begin{matrix}rowing & Mean Ankle Vertical Acceleration > - 8 \\ cycling or elliptical walking & otherwise\end{matrix}$$

FIG. 8C shows a feature space 800c that is defined by the forward swing proportion feature (defined above with reference to the y-axis in FIG. 8B) on the x-axis, and a trunk total acceleration RMS feature on the y-axis. In the feature space 800c, data points 802c (stars) correspond to a cycling activity and data points 804c (crosses) correspond to an elliptical walking activity. The trunk total acceleration RMS feature, which is the y-axis of the feature space 800c, can be defined as follows:

$$Trunk Total Acceleration RMS = \sqrt{\frac{{\sum }_{i = ts}^{te} {( TrunkTA ( i ) - {μ}_{TA} )}^{2}}{te - ts}}$$

where TrunkTA(i) is a trunk total acceleration (i.e., a Euclidean norm or magnitude of acceleration measured in three dimensions) generated at time i by an accelerometer attached to a trunk or waist area of a body, and where μ_{TA} is a mean of a trunk total acceleration measured over a swing cycle interval. The mean μ_{TA} may be calculated as follows:

$${μ}_{TA} = \frac{{\sum }_{i = ts}^{te} TrunkTA ( i )}{te - ts}$$

To simplify calculations without significant loss of accuracy, local acceleration due to gravity (g) may be substituted for RTA in the formula for the trunk total acceleration RMS feature:

$$Trunk Total Acceleration RMS = \sqrt{\frac{{\sum }_{i = ts}^{te} {( TrunkTA ( i ) - g )}^{2}}{te - ts}}$$

The RMS of trunk total acceleration represents an intensity of trunk motion. (To avoid the computational cost of a square root calculation, however, a mean square of trunk total acceleration can be used instead.)

The features defining the feature space 800c are useful in distinguishing, for example, cycling from elliptical walking. When cycling, the forward swing phase of a swing cycle interval is generally longer than the backward swing phase because the leg requires more force to push the pedal forward during a forward swing than to pull it back during a backward swing. As a result, cycling tends to have a forward swing phase that is more than half of an ankle swing cycle interval. Elliptical walking, on the other hand, has a shorter forward swing phase because a standing phase of elliptical walking is generally longer than the forward swing phase. As a result, elliptical walking tends to have a forward swing phase that is less than half of an ankle swing cycle interval. Thus, the forward swing proportion feature serves as a reliable discriminator for distinguishing cycling from elliptical walking. However, this feature alone might not be reliable when a cycling resistance or revolutions per minute is relatively low. Thus, the trunk acceleration RMS feature, which is generally lower for cycling than for elliptical walking, also serves as a reliable discriminator for distinguishing cycling from elliptical walking. Thus, the cycling and elliptical walking activities may be distinguished in the feature space 800c according to the following decision criteria:

$$Activity = \{ \begin{matrix}cycling & \begin{matrix}Forward Swing Proportion > 0.5 AND \\ Total Trunk Acceleration RMS < 1.1\end{matrix} \\ elliptical walking & otherwise\end{matrix}$$

FIG. 8D shows a feature space 800d that is defined by a trunk total acceleration at the end of swing feature on the x-axis, and a trunk total acceleration RMS feature (defined above with reference to the y-axis in FIG. 8C) on the y-axis. In the feature space 800d, data points 802d (diamonds) correspond to a walking activity and data points 804d (triangles) correspond to a running activity. The end of swing trunk total acceleration feature, which is on the x-axis of the feature space 800d, may be defined as follows:

Trunk Total Acceleration at End of Swing=TrunkTA(te)

where TrunkTA(te) is a trunk total acceleration (i.e., a Euclidean norm or magnitude of acceleration measured in three orthogonal dimensions) generated at the end of a forward swing phase of a swing cycle interval by an accelerometer attached to a trunk or waist area of a body.

The features defining the feature space 800d are useful in distinguishing walking from running. When walking, a double support event occurs at the end of a forward swing phase of a swing cycle interval in which both feet are supported on a surface. On the other hand, when a subject is running, a double float event occurs at the end of a forward swing phase of a swing cycle interval in which both feet are free-floating in the air. Thus, the double float event is a substantially zero gravity or low g event. The trunk total acceleration at end of swing feature is used to recognize when a trunk undergoes a double support event or a double float event at the end of a swing event and to thereby distinguish running from walking.

In addition, the trunk total acceleration RMS feature is an effective discriminator to distinguish walking from running because the speed of the trunk is generally higher when running than when walking. Thus, walking and running activities may be distinguished in the feature space 800d according to the following decision criteria:

$$Activity = \{ \begin{matrix}running & \begin{matrix}Total Trunk Acceleration RMS > \\ End of Swing Trunk Acceleration - 3\end{matrix} \\ walking & otherwise\end{matrix}$$

Referring again to FIG. 1, at 110 an act of identifying the activity based on one or more of the calculated features is performed. The activity identification can be performed in accordance with one or more of the decision criteria described above with reference to FIGS. 8A-8D. FIG. 4 demonstrates an order or priority in which features can be extracted and decision criteria applied. To preserve computational resources, calculation of features that are not required for classification might be omitted. Thus, for example, if the activity being identified is walking then the decision nodes 408 and 410 are not reached and the only features required to be calculated are those needed for the decision criteria at nodes 402, 406, and 412.

At 112, the identified activity can be used to monitor or assess the health of a subject engaged in the activity. For example, a fitness or health level can be assessed using a different metabolic rate, depending on the identified activity, to calculate an energy expenditure amount. A dynamic activity will have a higher metabolic rate than a static activity and high intensity dynamic activities will have higher metabolic rates than lower intensity dynamic activities. A table of metabolic rates can be accessed when assessing the fitness level. The fitness level assessment can include an estimation of energy expenditure based on the identified activity and based on characteristics of the activity, such as an activity speed, which can be derived from the swing cycle interval. Other non-health related applications might also use the identified activity, e.g., to enhance realism of a virtual-reality gaming or simulation environment.

The foregoing example embodiments can be used to classify motion as corresponding to an activity engaged in by a subject, such as a human body. The example embodiments can be used in conjunction with other methods and systems to identify more complex activities than those described above, to monitor health, to track performance of an exercise routine, to build medical histories, to detect health risks, and/or to augment a virtual reality system, among other things. In addition to the various alternative embodiments described above, various other versions of method 100 can be implemented including versions in which various acts are modified, omitted, or new acts added or in which the order of the acts differ.

For example, in the activity identification act 110, the decision criteria on which activity identification is based can be modified to account for variations in sensor outputs, such as differences in units of measurement, or other idiosyncratic characteristics of either the sensing devices or of the subject of observation. Moreover, the decision criteria can be modified to use non-linear decision boundaries that more accurately account for outlying data points in a feature space to avoid erroneous classifications. Alternatively, the processor 306 of the portable computing device 202 (or a processor in the computer 350 configured to receive the motion data and/or motion data features) can implement a trained neural network classifier optimized to receive motion data features and apply decision criteria to them based on a set of previously processed training features. In addition, the decision criteria, whether applied by a simple classifier or a trained neural network classifier, can be adaptive based on a history of data accumulated for a subject, such that classification is tailored over time to appropriately recognize any unique characteristics of the subject's particular motions.

The example embodiments disclosed herein may be embodied in other specific forms. The example embodiments disclosed herein are to be considered in all respects only as illustrative and not restrictive.

What is claimed is:

1. A method for classifying motion as corresponding to an activity type, the method comprising:
sensing motion characteristics associated with an activity using one or more motion sensors to generate a first set of data;
identifying a cycle interval in the first set of data; and
identifying the activity based on the interval.

2. The method as recited in claim 1, wherein the sensed motion characteristics include human limb motion characteristics.

3. The method as recited in claim 2, wherein the human limb is an ankle.

4. The method as recited in claim 1, wherein the one or more motion sensors include a gyroscopic sensor.

5. The method as recited in claim 1, wherein the activity is identified as one of a set of activities comprising: running, walking, rowing, cycling, and elliptical walking.

6. The method as recited in claim 1, further comprising:
sensing motion characteristics associated with the activity using the one or more motion sensors to generate a second set of data,
wherein the activity is identified based on the second set of data.

7. The method as recited in claim 6, wherein the motion characteristics sensed to generate the second set of data include at least one of trunk and ankle motion characteristics.

8. The method as recited in claim 6, wherein the one or more motion sensors includes an accelerometer, the second set of data being generated using the accelerometer.

9. The method as recited in claim 1, wherein identifying the activity based on the interval includes:
calculating a feature of motion data generated by a motion sensor during the interval; and
using the feature to identify the activity.

10. The method as recited in claim 9, wherein the first set of data includes the motion data generated during the interval, and
wherein the motion data includes angular velocity data and the calculated feature is an absolute magnitude of the angular velocity data.

11. The method as recited in claim 9, wherein the interval is an interval between consecutive occurrences of a forward swing event performed by a body part, and wherein identifying the activity is based in part on a duration time of the forward swing event.

12. The method as recited in claim 9,
wherein the first set of data includes angular velocity data characterizing angular motion of a first ankle, and
wherein the feature is an angular velocity feature calculated using at least a portion of the angular velocity data generated during the cycle duration.

13. The method as recited in claim 12, wherein the activity is identified based on whether the angular velocity feature exceeds a first threshold.

14. The method as recited in claim 12, further comprising:
sensing vertical acceleration characteristics of at least one of the first and a second ankle using the one or more motion sensors to generate vertical ankle acceleration data; and
calculating a vertical ankle acceleration feature using at least a portion of the vertical ankle acceleration data generated during the interval,
wherein the activity is identified as rowing based on the angular velocity feature and the vertical ankle acceleration feature.

15. The method as recited in claim 14, further comprising:
sensing acceleration characteristics of a trunk portion of a body using the one or more motion sensors to generate trunk acceleration data;
calculating a trunk acceleration measurement using at least a portion of the trunk acceleration data generated during the interval; and
calculating a forward swing proportion feature using at least a portion of the angular velocity data generated during the interval, the forward swing proportion measurement being indicative of a proportion of the interval that corresponds to a forward swing motion,
wherein a cycling activity is distinguished from an elliptical walking activity based on the angular velocity feature, the trunk acceleration feature, and the forward swing proportion feature.

16. The method as recited in claim 12, further comprising:
sensing acceleration characteristics of a trunk portion of a body using the one or more motion sensors to generate trunk acceleration data;
calculating a first trunk acceleration feature using at least a portion of the trunk acceleration data generated during the interval; and
calculating a second trunk acceleration feature using the trunk acceleration data generated at a single time in the interval,
wherein a walking activity is distinguished from a running activity based on the angular velocity feature and the first and second trunk acceleration features.

17. One or more computer-readable media having computer-readable instructions thereon which, when executed, implement a method for classifying human motion as corresponding to an activity, the method comprising the acts of:
sensing motion characteristics associated with the activity using one or more motion sensors to generate a first set of data;
identifying a cycle interval in the first set of data; and
identifying the activity based on the interval.

18. A system for classifying motion as corresponding to an activity, the system comprising:
a memory configured to store motion data;
a processing circuit configured to carry out the following acts:
sensing motion characteristics associated with the activity using one or more motion sensors attached to a subject to generate a first set of motion data;
storing the motion data in the memory;
identifying a cycle interval in the first set of motion data; and
identifying the activity based on the interval.

19. A method for assessing fitness of a human subject, the method comprising:
sensing characteristics associated with activities engaged in by the subject using one or more sensors attached to predetermined positions on the subject;
identifying a cyclical pattern in at least one of the sensed characteristics;
identifying the activities based on the sensed characteristics and the cyclical pattern; and
assessing a fitness of the subject based on the identified activities.
